(19) United States
(12) Patent Application Publication
(10) Pub. No.: US 2020/0384240 A1
(43) Pub. Date: Dec. 10, 2020

(54) LIGHTING DEVICE, METHOD AND PROGRAM FOR COUNTERING MOTION SICKNESS EXPERIENCED IN A VEHICLE

(71) Applicant: OSRAM GmbH, Munich (DE)

(72) Inventors: Norbert Linder, Lappersdorf (DE); Norbert Haas, Langenau (DE)

(73) Assignee: OSRAM GmbH, Munich (DK)

(21) Appl. No.: 16/893,401

(22) Filed: Jun. 4, 2020

(30) Foreign Application Priority Data
Jun. 6, 2019 (DE) 10 2019 208 273.4
Jul. 26, 2019 (DE) 10 2019 211 182.3

(51) Int. Cl. A61M 21/02; B60Q 3/70; B60Q 3/80

(52) U.S. Cl. CPC A61M 21/02; A61M 2021/0044; B60Q 3/80; B60Q 3/70

(57) ABSTRACT

A lighting device (2) mitigating motion sickness experienced by an occupant in a moving vehicle having at least one light source (90) and an operating unit (60, 70, 80, 120, 130) for recording data of a vehicle movement and/or a control signal that corresponds thereto, wherein the light source (90) outputs a light pattern on the basis of the recorded data and/or of the control signal, depending on a speed, an acceleration, a change in the acceleration and/or a change in direction of the vehicle in a respective axial component in the direction of travel as well as a transverse component orthogonal thereto.

### CROSS-REFERENCE TO RELATED APPLICATIONS

This application claims priority to German Patent Application Serial No. 10 2019 208 273, which was filed Jun. 6, 2019, and to German Patent Application Serial No. 10 2019 211 182, which was filed Jul. 26, 2019, each of which is incorporated herein by reference in its entirety.

### BACKGROUND

The topic of motion sickness, also known as kinetosis or vertigo, plays a role, in particular in connection with fast-moving or accelerating vehicles, in particular cars, since physical and mental malaise can be triggered through effects related to lateral and transverse movement and acceleration. This unwanted effect can arise in particular with the advent of partially and fully automatically driving vehicles in which a driver no longer has to intervene in the driving process at all, or only must do so partially, and therefore no longer has to pay attention to the driving environment. The same also applies to passengers. Such effects can also occur when using automobiles, aircraft, ships, trains and the like.

According to one explanatory theory, motion sickness is caused by an inconsistency between the pattern of sensory input values and the expected body-related input values, or as an inconsistency between different sensory input values. The organ of balance thus perceives speeds and their changes that the eye does not perceive if it is not oriented to the road. According to another explanatory concept, motion sickness is provoked by a loss of body-related control.

Patent Application Pub. US2017/0291538 (Sivak) describes in this context a light unit that is arranged in the peripheral field of view of a passenger and is operated in such a way that the physiological effect of the light signal counteracts that of the vestibular system, and thus avoids motion sickness. The light unit is operated here in such a way that the lighting simulates what the affected person would see if they were to look outside from the vehicle. The light matrix can be operated in such a way that the alignment of the vehicle, that is the roll, pitch and yaw angle, as well as the speed and the acceleration, affect the light pattern that is to be displayed. The light matrix can be attached directly to the head of the driver as a wearable unit, or permanently installed in the vehicle.

Patent Application Pub. US2018/0086258 (Fleurence) describes a light unit that can radiate dynamic light patterns along or across the direction of the vehicle axis. The dynamic movement of the light pattern can here correspond—at least proportionally—to the speed and acceleration of the vehicle.

Patent Application Pub. US2018/0161537 (Ketels) describes a signal apparatus that signals the change of vehicle data, for example when entering a bend to the right or left, or a change in the speed of the vehicle. In response to an increase in the speed, the running light pattern runs from the rear to the front, and when braking from the front to the rear.

While dynamic light patterns are accordingly at present generated and displayed to prevent motion sickness, these do not however take the directional components of the perceived vehicle movements into account.

### DESCRIPTION OF PREFERRED EMBODIMENTS

Present embodiments herein provide a lighting device for a vehicle through which motion sickness can be better prevented, or which counters, mitigates or at least minimizes motion sickness it if should it occur. The provision of a vehicle with a communication interface for such a lighting device is also provided. A method for the output of a light pattern in a vehicle is provided, as well as a computer program for carrying out the method is also provided.

Further advantageous developments of the present embodiments are described in the dependent claims.

The starting point is a lighting device with at least one lighting means for a vehicle, wherein the lighting means device comprises an operating unit for recording data of a vehicle movement and/or control signals that correspond thereto.

The operating unit can comprise sensors or other detection units in order to acquire data of a vehicle movement. The operating unit can, alternatively or in addition, also comprise a communication interface via which corresponding data are transmitted to the operating unit. The data can then be converted by the operating unit into control signals by means of which at least one lighting means is driven for the output of a light pattern. In this case, the lighting device comprises a control unit that is included in the operating unit or which is connected to it for signaling. Alternatively or in addition, the operating unit can also directly record external control signals and forward them to the at least one lighting means, or also adapt them. The term “record” is thus not restricted to the acquisition of data, but is also to be understood in the sense of signal transmission.

According to the present embodiments, the light source 90 outputs a light pattern on the basis of the data and/or of the control signals, depending on a speed, an acceleration, a change in the acceleration and/or a change in direction of the vehicle in a respective axial component in the direction of travel, as well as a transverse component orthogonal thereto.

The parameters of speed, acceleration, change in acceleration and/or change in direction that represent the vehicle movement here from the point of view of motion sickness can, as already mentioned, be acquired at the time by corresponding sensors. A detection unit for provision of the parameters can, however, also, alternatively or in addition, be realized through an anticipation unit. An anticipation unit generates corresponding vehicle movement data on the basis of a predicted road path and the boundary conditions associated therewith such as average speeds, maximum speeds, detected traffic participants etc. Current data regarding the vehicle movement, on the basis of which, changes to the speed and/or acceleration can be determined with greater precision, can be further boundary conditions. In one embodiment, the anticipation unit obtains the data and the boundary conditions for calculation from a navigation system, or as a component of said system. This form of digital mapping for the generation of control data for the light pattern that is to be output can be optimized through deep-learning methods or AI applications.

The parameters of speed, acceleration, change in the acceleration and/or change of direction of the vehicle movement in a respective axial component in the direction of travel, as well as in a transverse component orthogonal thereto, in particular a transverse component parallel to the roadway, condition the light pattern that is to be output. Since bodily perception is not one-dimensional, a light pattern that can be associated with a corresponding two-dimensional bodily impression, or a to corresponding bodily impression can also be output by this means.

Experimental measurements have shown that the acceleration (B), or, respectively, the change in the acceleration (B′) of a vehicle, is largely responsible for causing motion sickness. It has been shown that it is advantageous if the light pattern, light colors, and dynamic changes to the light pattern that are to be set are a function (or functions) of the acceleration (B) or of the change in the acceleration (B′), i.e. according to the relationship that:
Light pattern, light colors, or dynamic change of the light pattern=K*functions (B, B′), where K=correlation factor.

The axial components in the direction of travel of the vehicle (BA, BA′) and a transverse component orthogonal thereto (BQ, BQ′) are to be considered here. The functions can generate periodic light patterns, for example sinusoidal or sawtooth-like with return function. Stochastic light patterns can also be generated.

The changes to these acceleration values, i.e. (BA′) and (BQ′), are included as separate variables in the generation and control of the light patterns. In other words, a change in the drive of the lighting means (e.g. running frequency, light flash frequency, color change) occur as functions of (BA′) and (BQ′). The respective functional correlation factor (KA, KA′, KB, KB′) does not have to be constant, but can change in a linear or non-linear manner with changing values (KA, KA′, KB, KB′). The sum values and products of the values (KA, KA′, KB, KB′) can also be used as control signals. It follows from this that the respective functional correlation factors KX, where X represents the respective parameters of the vehicle movement, can differ. Correlation factors that are different in each case, depending on the directional component of the respective parameter, are also possible.

The light pattern to be output is to be radiated into an interior of the vehicle for perception by vehicle occupants. An interior vehicle light presents itself for this purpose. It is, however, also conceivable that lighting devices or lighting means are arranged outside the interior of the vehicle, provided light signals can still be registered in the vehicle interior, for example through vehicle windows.

The lighting means, or rows of lighting means, are accordingly arranged in the vehicle such that they can be perceived by the occupants. It is sufficient for this purpose if they can be detected in the horizontal or vertical peripheral field of view. The lighting means can here be connected permanently to the vehicle, or integrated into components of the interior fittings, for example in the ceiling of the passenger compartment, or at the side fittings, at the seats, on the floor, on the pillars, as well as at or in the windows, or only introduced in a transportable manner into the interior and then removed again. The lighting means can be arranged in the axial direction of the vehicle or transversely to that, in particular perpendicular to the axial direction. The lighting means can also extend in a vertical direction.

Linear or curved light strips that are formed of a large number of lighting means can be used to output the light patterns. The lighting arrangements can be installed permanently into the vehicle, or may also be attached subsequently. Examples of the latter are interior lights that can be attached in a vehicle, for example operated by batteries, such as the adjustable-color light strips sold under the trade designation “OSRAM LEDambient”, or such as an attachable orientation and reading light, such as that sold under the trade designation “OSRAM COPILOT”, that can be attached in a vehicle, or such as standard commercial LED magnetic lights. Adjustable-color, transportable lighting means or lamps, such as for example those available from OSRAM under the trademark LIGHTIFY™ or under the trade designation “Philips COLOR HUE” can, perhaps after modification, also be introduced to a vehicle interior and placed and operated there. Light-emitting textiles, for example work clothing provided with lighting elements, sport or leisure clothing, or items of outdoor equipment provided with strip-shaped lighting devices can also be introduced into a vehicle, placed and operated there. The lighting means can be arranged in one row or multiple rows, for example in 10 rows each having 50 separate lighting means. These can extend substantially in parallel. The lighting means can also, however, form matrix-like arrangements, wherein the mutual distances do not have to be constant.

Groups of lighting means can be arranged here in such a way that their sum color value generates a white impression, i.e. runs on or close to the Planck curve of the CIE chromaticity diagram. Some or all of the lighting means of a row or of multiple rows can be implemented with the same color. The lighting means can be arranged in groups. A large number of arrangements are conceivable here. The respective lighting means or groups of lighting means are connected to operating devices such as an electrical supply device or a control device. A vehicle can make direct or alternating current available.

OLED light sources, LEDs, mini-LEDs and micro-LEDs can be used as lighting means. In particular when using mini-LEDs or micro-LEDs, a large number of lighting means can be used, for example several thousand. The light arrangement itself can be implemented as a woven textile, i.e., for example, as flat light guides, for example as tapes or fibers worked into a textile weave. This textile implementation is designed as a vehicle interior light for generating and representing at least some of the above-mentioned functions.

The light strips or light arrangements are designed not only to generate static lighting effects, but also dynamic lighting effects.

Preferably, the at least one lighting means can output light in different colors and/or at least two lighting means that output different light are provided.

Through differently colored light that can be output by a lighting means, a plurality of lighting means and/or groups of lighting means, the lighting device can be operated in such a way that a specific bodily impression is underscored by color, or also that additional information is visually displayed by the color. For example, a plurality of lighting means or groups of lighting means can, at a slow running frequency, i.e. a serial light sequence at a predetermined frequency, output light of a first color, e.g. green, and at another, for example faster, running frequency at least some other lighting means emit light of a second light color, red for example.

In one embodiment, the lighting device is designed such that the light pattern can be represented in a horizontal direction over an angular range of approximately 180° to 200° and/or in a vertical direction over an angular range of about 130°, and that preferably at least one light output parameter, in particular a light color, can be changed over this range.

The stated angular ranges cover the peripheral field of view of a vehicle occupant, and thus enable a high degree of coverage. In this coverage range, the lighting means can be controlled in such a way that the lighting means lying further out in the peripheral field of view of the vehicle occupant emit light of a first kind, for example of a yellow color, then the lighting elements lying inward in the peripheral field of view of the vehicle occupant emit light of a second kind, for example of a blue color, and then, yet further inward, in a third kind of light, for example of a reddish color, and finally a fourth kind of light, for example of a greenish color. Said kind of light is not restricted to colors, but can also relate to the size of the light spot, the intensity of the light, a flashing or running frequency or other differences in the light signal. The kinds of light can themselves form a light pattern, or different kinds of light can be combined into a light pattern.

Light type settings can also be made in relation to the peripheral angular ranges of the near region, middle region and far region. In other words, for example, in a running process proceeding from outside inwards, the respective activated light means or groups of light means also change their light color in addition to their movement light pattern. The same applies, although with the opposite sequence, if the light pattern runs from inside outward. In a further aspect, the light means are controlled depending on the relative orientation of vehicle occupants and vehicle interior lights, wherein the respective lighting means of a lighting device can be driven differently in terms of, for example, intensity, connection in a group, and flashing frequency.

The coverage region referred to for the perception of light signals in the peripheral field of view is related to an ideal alignment of lighting device and vehicle occupants to one another. The possibility of adjusting the alignment will be considered later.

In an advantageous development, the light pattern has a running direction, and the light intensity and/or the color of the light can be controlled depending on the running frequency.

The running direction results from the serial drive of lighting means or lighting means groups that are arranged in a running direction. Light patterns that run in a linear manner, i.e., for example, from the front (in the direction of travel) to the rear, or from right to left, or vice versa in each case, are significant, since they can display the current direction of travel, as well as reproducing light patterns or flow sequences generated from the current acceleration or the current change in the accelerations. Light patterns that run from above, i.e. from the roof, downward to the vehicle floor and vice versa can also be generated. The light patterns can be repetitive, wherein the repetition rate can be a function of the speed, the acceleration and/or of the change in the acceleration of the vehicle.

The lighting means of the lighting device that form the running pattern can additionally be changed in their intensity, for example with a higher light intensity at higher running frequencies, or with a lower light intensity as the running frequency becomes slower. The lighting means can also be operated with a flashing frequency, and this can also be true when in a stationary state as well as with a dynamically adjusted light pattern. Depending on specification or setting, this can then also change, for example also flashing more quickly as the vehicle becomes faster.

This relates both to the drive of a running pattern in the axial direction of the vehicle as well as transversely thereto. In one variant, the running direction of a pattern is configured in the direction of the centrifugal force, i.e., when for example turning to the left, the running pattern would move towards the right, i.e. in the direction opposite to that of the turn.

The running direction of the light pattern thus corresponds to the bodily perception, while the light intensity or color enhances the impression depending on the running frequency. Running frequencies are here preferably raised with increasing speed. The increase in risk associated with increasing speed can be visually displayed through higher light intensities and/or the use of signal colors such as red. An improved perceptibility can also be achieved, through which the motion sickness, which is frequently intensified at higher parameter values of the vehicle movement, can be more effectively countered.

The operating unit is preferably designed such that the color location of the light pattern in the CIE chromaticity diagram can be adjusted to an ambient light.

The light color of the light source or light sources of the lighting device, or, put more precisely, its respective color location in the CIE chromaticity diagram, is changed in such a way that the emission curve or the spectrum of the sum of the emitted light or of the operated light sources exhibits, for example, a best possible match to the current daylight spectrum as it is formed by solar radiation through clouds and the atmosphere, known as circadian light, and/or the best possible match of the individual color components of the respective light lighting means, in particular in the red, green and blue spectral range, to the sensitivity curve of the rod-shaped photoreceptors. This also concerns dusk or nighttime driving, in order to achieve a best possible match to the sensitivity curve of the rod-shaped photoreceptors in the range of dusk and night vision. Green color tones in particular are particularly easily perceived at night. Further variations are conceivable.

In one embodiment, the at least one lighting means is designed for the output of infra-red radiation.

Through the use of infra-red radiation, light patterns can be made available in the form of flashes, in particular in the form of double flashes (multi-photon effect); a wavelength of up to about 1000 nm is perceptible. Infra-red radiation can thus also contribute to minimizing motion sickness. Inter alia, the advantage lies in that the perception of the vehicle occupants is influenced by the infra-red radiation, while the perception of other traffic participants is scarcely distorted thereby.

In one advantageous development, the light pattern can be adjusted depending on user specifications and/or biodata.

A user can accordingly set the configuration of the light pattern in the sense of running direction, frequency, color etc. according to the user's own specifications such as user experience, age, body-related conditions, eye-specific data or medical impairments. The lighting apparatus can be permanently or wirelessly connected for this purpose to an input device that the user can operate and thus convey the input data to the operating unit of the lighting device or a control unit for the operating unit.

Alternatively or in addition, the operating unit or the control unit can also record biodata of a vehicle occupant, such as pulse rate, blood pressure, electrical skin resistance, the blinking of an eye, a seating position etc. The biodata are converted into a control sequence for a light pattern, and made available to the operating unit of the illumination device or of the control unit which is part of the lighting device, or the data are transmitted to this device. The lighting device can, alternatively, also calculate the control sequence data itself.

The lighting device preferably comprises an orientation detection unit that acquires the orientation of the lighting device with reference to the vehicle and/or a vehicle occupant, wherein the light pattern can be aligned to this orientation.

Near-field communication (NFC) devices are, for example, arranged at various positions in the vehicle interior such as the footwell, at side pieces, on the roof or at the vehicle seats, and when the lighting device, whose orientation detection unit is also NFC-enabled, is put into place, said devices then transmit a position signal to the orientation detection unit which can be stored by said unit. Alternatively or in addition, the lighting device, or the orientation detection unit, is designed to communicate with a user and/or the vehicle. The position signal is received by the orientation detection unit of the lighting device, and, in a computing unit, for example the operating unit, processed as a confirmation signal of an orientation position associated therewith, and forwarded to the control unit for the light signal. The position and orientation of the lighting device, or the position of the light-generating lighting means, is thereby known, and thus also the position and orientation with respect to one or a plurality of users for vehicle occupants. It is, in principle, also conceivable that a lighting device can communicate with external communication devices that are located outside the vehicle.

It is also conceivable that the position and orientation of the vehicle interior light is determined by means of triangulation techniques. The orientation detection unit can transmit, for example, radio waves, infra-red radiation or acoustic waves, in particular in the ultrasonic range, for this purpose, in order thus to make it possible for the position and alignment of the lighting device to be determined. The position of a user can, for example, be identified by means of the seating position that has been set and/or by means of interior cameras. A correlation between the user orientation and the lighting device is thereby established. It is also conceivable that the position of an occupant, in particular the head position and thus the field of view currently being perceived, is determined continuously, for example by an interior camera or an interior LIDAR system for face recognition. In this way it is made possible to carry out a dynamic lighting control adapted to the current situation of one or a plurality of lighting devices.

In addition to the drive of the lighting means for the output of the light pattern, it is possible for the control device to regulate the driving behavior of the autonomously driving vehicle in such a way that the values for acceleration or for change in the acceleration are kept low. These values can, as described above, also be actively changed by a vehicle occupant, also set or changed automatically using biofeedback.

With appropriate design, the lighting device can also serve to cover other customer usage functions, in particular when being carried in an autonomous vehicle such as, for example, a display of the light color of a traffic light through recording by the vehicle camera, or a display of an external flash or of a brake light.

The lighting means arrangement or grouping can also be used for the display of graphic patterns such as smileys, generally understandable logos and information symbols, as well as for text messages generated by the vehicle, such as: Everything okay; Now driving onto the freeway; Reaching destination in 5 minutes; Do you want to do any more shopping? A text display to request an information input by means of voice or another medium such as a mobile telephone connected, in particular wirelessly, to the vehicle interior lights or the vehicle, is also possible.

Other possible displays are, for example, the graphic display of the battery charge level, the fuel filling level, an emergency warning indicator, information supplied from outside, a direction display for external events such as a police siren or vehicles passing by that are optically or acoustically measured with external cameras or microphones and then displayed in the appropriate direction in the interior graphically and/or by means of light color. Such a lighting device can also be used for regular, in particular adjustable, interior lighting, wherein the interior lighting function is switched off at specific threshold values of the parameter values of the vehicle movement, in particular at specific threshold values of the acceleration and/or change in the acceleration, and which then reproduces the light pattern for minimizing the motion sickness. Independently of the use of the lighting device for regular interior lighting, it is also in principle possible to provide that the lighting device is only activated above a minimum speed or of the parameter values for an acceleration and/or change in the acceleration.

It is further conceivable that the lighting device and/or the vehicle interacts directly or indirectly with the mobile device, smartphone, tablet, e-book reader, laptop of a user and, using control commands transmitted by Bluetooth or wirelessly, similar color patterns, color sequences or instructions, in particular warning instructions are displayed by the mobile device, for example as a colored warning point, dotted line or pattern at one or both sides of a smart phone, tablet or the like. In the presence of axial acceleration, these color symbols can proceed at one or both display sides, with transverse acceleration at only one side, for example only in the direction of turning or only in the direction of the centrifugal force that acts on the vehicle or occupants. The intensity of the symbols can, for example, be changed in such a way that it increases with increasing agreement between the display alignment and the axial or transverse direction.

Due to its position sensors, a smartphone can recognize its own alignment, as well measure its own acceleration by means of acceleration sensors, and can thereby generate the corresponding light signals itself, and thus itself represent a lighting device. The control commands can, alternatively, also be transmitted to the mobile device by radio or Wi-Fi.

In a further aspect, present embodiments also relate to a vehicle with a communication interface for a lighting device described above, wherein the communication interface is configured to convey data dependent on vehicle movement and/or occupants, position and/or orientation data of the lighting device, and/or control signals.

As a result, the lighting device does not itself have to comprise all the detection or control units necessary to carry out its functions, but can make use of the vehicle's equipment. In particular, data that has already been determined by the vehicle for other reasons does not have to be separately acquired.

The communication interface can also be used so that the driving behavior of autonomously driving vehicles is regulated in accordance with prescriptions of the lighting device in such a way that the parameter values of the vehicle movement, in particular values for the acceleration or for the change in the acceleration, do not exceed predetermined threshold values. These values can, as described above, also be actively changed by a vehicle occupant, also set or changed automatically using biodata.

Present embodiments also relate to a method for outputting a light pattern in a vehicle, comprising the steps of:
a) determining data relating to vehicle movement and/or occupants;
b) choosing a light pattern in accordance with the determined data; and
c) driving a lighting means or light source in accordance with the selected light pattern,
wherein the determining data (step a)) is preferably carried out continuously in order to adapt the light pattern on the basis of the determined data.

The data are determined on the basis of currently detected and/or anticipated parameter values and/or user inputs. The parameter values of the vehicle movement are determined in their respective directional components. The selection of the light pattern to be output depending on the determined data results from the application of an algorithm for calculating concrete light patterns and/or through a comparison with threshold values and light patterns stored for this purpose. The drive signals provided for this purpose emerge from the selection of the light pattern.

Through the continuous determination of the data in step a) the light pattern is continuously adapted to the given boundary conditions. Preferably, however, an adaptation only takes place when a change is determined in at least one of the parameter values lasting over a predetermined duration, for example 1 s to 5 s. This stops an excessively high change frequency occurring which could negatively influence the well-being of a vehicle occupant. Short-term outliers thus also are not considered.

A further aspect of present embodiments relates to a computer program for storage on a data carrier for carrying out the above-described method, comprising the steps of:
a) acquiring data relating to vehicle movement and/or occupants,
b) choosing a light pattern in accordance with the determined data, and
c) driving a light source in accordance with the selected light pattern.

The acquisition of the data can comprise initiation of a determination of the data, or may also only be aimed at the recording of appropriately determined values. The computer program can be stored for this purpose in the lighting device, or may be implemented on an external control device for driving the lighting device.

A lighting device and/or a mobile device such as a mobile phone, smartphone, tablet, laptop, e-book reader etc., or also a mobile device acting as a lighting device can comprise the computer program, for example a downloadable app from an app store that captures internal sensor data as input values and from then, in accordance with adjustable control functions, calculates light control signals and then transmits these to the lighting metal controller for execution. Due to its position sensors, a smartphone can recognize its own alignment as well measure its own acceleration by means of acceleration sensors, and can thereby generate the corresponding light signals in accordance with program specifications. The software app is preferably implemented as a user interface, and is stored in a non-volatile, computer-readable medium from where it can be called.

Further advantages, features and details of the embodiments emerge from the claims, the description of preferred embodiments, and with reference to the drawings. Like reference signs in the figures identify like features and functions.

### BRIEF DESCRIPTION OF THE DRAWINGS

FIG. 1 shows a cross-sectional view (perpendicular to the direction of travel) of a vehicle with lighting devices according to a first exemplary form of embodiment.

FIG. 2 shows a block diagram for the drive of a light pattern.

### DETAILED DESCRIPTION OF PREFERRED EMBODIMENTS

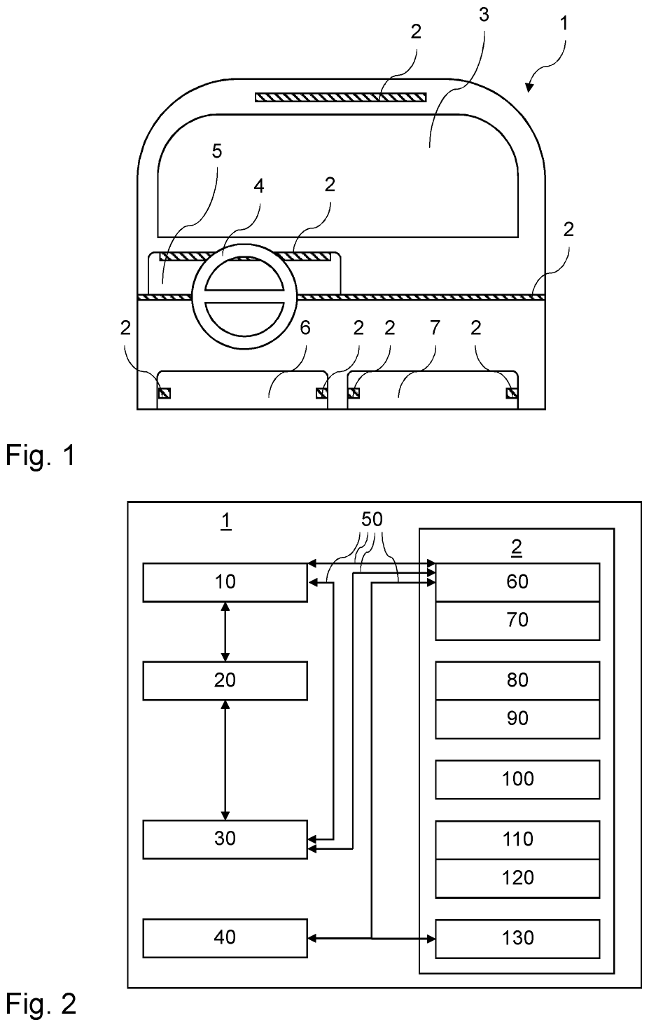

FIG. 1 shows a cross-sectional view of a vehicle 1 with lighting devices 2 according to a first exemplary form of embodiment. The cut plane extends perpendicularly to the direction of travel of the vehicle 1, and is located at the height of the seat surfaces of the driver's seat 6 and of the passenger seat 7, wherein the direction of view is shown in the direction of the windscreen 3. Even though the present embodiment is primarily oriented to the minimization of motion sickness when using automatic driving systems, for the sake of better orientation the terminology from the field of conventional vehicles, to which the present embodiment is likewise also applicable, is adopted.

The interior of the vehicle comprises a plurality of lighting devices 2 that are arranged in various regions of the vehicle 1. In the schematically illustrated example, the lighting devices 2 are located above the windscreen 3, in the upper edge region of the instrument cluster 5, below the instrument cluster 5, over the entire width of the vehicle, and at each of the side seating surfaces of the driver seat 6 and of the passenger seat 7. Further lighting devices 2 may be provided that can also be integrated, for example, into an armrest, other kinds of supporting element, a display holding component or a shelf for objects, and in particular extend at the sides in order to at least not fully cover the lighting means or light source(s) 90 of the lighting device 2 when in use.

The lighting devices 2 illustrated in FIG. 1 are integrated into the vehicle 1, but can, however, also be fastened or removably arranged as mobile lighting devices at appropriate locations. Positive-locking, friction-locking or other known fasteners can be applied for this purpose. For example hook-and-loop closures, such as those conventionally sold under the trade designation Velcro™ or magnetic systems that enable easy connection and release are preferably employed.

FIG. 2 shows a block of a drive variant for the output of the light pattern to minimize motion sickness. The vehicle 1 comprises for this purpose a vehicle controller 10, a control unit 20 and at least one near field communication device 30, as well as at least one lighting device 2. The double arrows represent radio or cable connections 50. The lighting device 2 here is a mobile lighting device, and comprises a communication interface 60, a data store 70, a light source driver 80 as well as associated light source or light sources 90, an energy supply 100 in the form of a battery unit or mains connection for a 12 V or 24 V DC supply or a 230 V alternating voltage provided by a voltage converter, a detection unit 110, a control unit 120 and a user interface 130. The communication interface 60 comprises transmitting and receiving devices for communication over WLAN, Bluetooth and NFC protocols, as well as for voice control. The data store 70 comprises a data storage unit as well as software and operating programs that contain sequence patterns, logos, text messages, operating parameters and light programs. The light source(s) 90 are driven by the light source driver 80 which is in connection with the light source 90 as well as the detection unit 110 and actuators such as an ultrasonic sensor and receiver. The detection unit 110 here comprises internal sensors for measuring the acceleration as well as the ambient brightness. In the exemplary embodiment illustrated, the detected parameter values are not directly processed by the light source driver 80, but are first converted into drive signals by the control unit 120. A vehicle occupant 40 can also make further specifications that have an influence on the drive of the light source 90 via the user interface 130. The user interface 130 for example allows the selection of the parameters that determine the light pattern, so that, for example, light patterns are only output on the basis of a change in the acceleration, and the specification of the respective parameter threshold values. In a similar way, the vehicle occupants 40 can also have an influence on the drive of the light source 90 via the communication interface 60.

The mobile lighting device 2 is arranged in the vehicle interior, and receives a position signal of the near field communication device 30, through which a position and orientation of the lighting device is determined. The vehicle controller 10 can additionally transmit further sensor and operating data via the communication interface 60 to the light source driver 80. Additional data is made available in this way, and/or a monitoring comparison of the sensor data of the vehicle 1 with the lighting device 2 is enabled. The vehicle controller 10 can also contain data prepared by the control unit 20 according to a computing, storage or evaluation unit contained in the control unit 20 or transmit direct control commands.

The embodiments are not restricted to any particular example, but include further variants or combinations. In addition to optical signals, acoustic signals can thus also be provided, with which frequencies and tone sequences, or the excitation of a loudspeaker of an arrangement of loudspeakers, can also be calculated by the above-mentioned control functions and made available as a data set.

### LIST OF NON-LIMITING REFERENCE NUMERALS

1 Vehicle
2 Lighting device
3 Windscreen
4 Steering wheel
5 Instrument cluster
6 Driver's seat
7 Passenger seat
10 Vehicle controller
20 Control unit
30 Near-field-communication-device (NFC)
40 Vehicle occupant
50 Radio or cable connection, NFC
60 Communication interface
70 Data memory
80 Light source driver
90 Light source or lighting means
100 Energy supply
110 Detection unit
120 Control unit
130 User interface

What is claimed is:

1. A lighting device (2) for a vehicle (1), the lighting device (2) comprising:
at least one light source (90); and
an operating unit (60, 70, 80, 120, 130) for recording data of a vehicle movement in a travel direction and/or at least one control signal corresponding thereto; and
wherein the operating unit comprises a light source driver (80) configured to drive the at least one light source (90); and
wherein the light source (90) outputs a light pattern on the basis of the recorded data and/or of the control signal, depending on at least one of a speed, an acceleration, a change in the acceleration and/or a change in direction of the vehicle in a respective axial component in the travel direction as well as a transverse component orthogonal thereto.

2. The lighting device (2) of claim 1, wherein the at least one light source (90) is configured to output light in a plurality of colors and/or at least two lighting means (90) that output different light are provided.

3. The lighting device (2) of claim 1, wherein the light pattern can be represented in a horizontal direction over an angular range of about 180° to about 200° and/or in a vertical direction over an angular range of about 130°, and that at least one light output parameter can be changed over the angular range.

4. The lighting device (2) of claim 1, wherein the light pattern has a running direction, and the light intensity and/or the color of the light can be controlled depending on a running frequency from the light source driver (80).

5. The lighting device (2) of claim 1, wherein the operating unit (60, 70, 80, 120, 130) is configured to adjust, within the CIE chromaticity diagram, a color locus of the output light pattern to match a detected ambient light.

6. The lighting device (2) of claim 1, wherein the at least one light source (90) is configured to output infra-red radiation.

7. The lighting device (2) of claim 1, wherein the light pattern can be adapted depending on a user specification and/or biodata.

8. The lighting device (2) of claim 1, wherein the lighting device (2) comprises an orientation detection unit (60, 120, 130) that acquires a spatial orientation of the lighting device (2) with reference to the vehicle (1) and/or a vehicle occupant (40), and the light pattern can be aligned to the spatial orientation.

9. The lighting device (2) of claim 1 in combination with the vehicle (1), the vehicle comprising a communication interface (10) adapted to interface with the lighting device (2), wherein the communication interface (10) is configured to transmit a vehicle movement data and/or a data indicative of a vehicle occupant, a position and/or orientation data of the lighting device (2), and/or a control signal.

10. A method for outputting a light pattern in a vehicle (1), comprising:
determining data indicative of a vehicle movement and/or a vehicle occupant;
selecting a light pattern in accordance with the determined data; and
driving a light source (90) in accordance with the selected light pattern;
wherein said determining data is carried out continuously in order to adapt the light pattern dependent upon the determined data.

11. A computer system comprising one or more non-transitory computer readable memories which store, in combination or singularly, instructions that, when executed by a one or more computers, cause the one or more computers to perform operations for adjusting a light pattern of a tunable light source (90), said operations comprising:
acquiring data relating to a vehicle movement and/or a vehicle occupant;
selecting a light pattern in accordance with the acquired data; and
driving a lighting means (90) in accordance with the selected light pattern.

12. The lighting device (2) of claim 1, wherein the at least one light source (90) comprises at least two said light sources (90) configured to emit light of a color different from each other.

13. The lighting device (2) of claim 3, wherein the at least one light output parameter is a light color.

14. The lighting device (2) of claim 1, wherein the light source (90) is driven by the light source driver (80) to output a light pattern dependent on the acceleration and/or the change in acceleration in direction of the vehicle in the respective axial component in the travel direction as well as the transverse component orthogonal thereto.
